SEQUENCE LISTING (7 sequences)

SEQ ID NO: 1 - protein, 349 aa, Synthetic construct
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEPIYMTGFEENAMWKLRADLPRIIPDAAIYYGYMYGMSALKIFAGFLFVDTWQYFLHRLMHMNKTLYKWFHSVHHELYVPYAYGALFNNPVEGFLLDTLGTGIAMTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

SEQ ID NO: 2 - protein, 349 aa, Synthetic construct
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEPIYMTGFEENAMWKLRADLPRIIPDAAIYYGYIYGMSALKIFAGFLFVDTWQYFLHRLMHMNKTLYKWFHSVHHELYVPYAYGALFNNPVEGFLLDTLGTGIAMTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

SEQ ID NO: 3 - protein, 349 aa, Synthetic construct
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEPIYLTGFEENAMWKLRADLPRIIPDAAIYYGYIYGMSALKIFAGFLFVDTWQYFLHRLMHMNKTLYKWFHSVHHELYVPYAYGALFNNPVEGFLLDTLGTGIAMTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

SEQ ID NO: 4 - protein, 349 aa, Synthetic construct
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEPIYMTGFEENAMWKLRADLPRIIPDAAIYYGFLYGMSALKIFAGFLFVDTWQYFLHRLMHMNKTLYKWFHSVHHELYVPYAYGALFNNPVEGFLLDTLGTGIAMTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

SEQ ID NO: 5 - protein, 349 aa, Synthetic construct
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEPIMMTGYEENAMWKLRADLPRIIPDAAIYYGYYYGMSALKIFAGFLFVDTWQYFLHRLMHMNKTLYKWFHSVHHELYVPYAYGALFNNPVEGFLLDTLGTGIAKTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

SEQ ID NO: 6 - protein, 349 aa, Synthetic construct
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEIIYMTGYEENAMWKLRADLPRIIPDAAIYYGYMYGMSALKIFAGFLFVDTWQYFLHRLMHMNKTLYKWFHSVHHELYVPYAYGALFNNPVEGFLLDTLGTGIAMTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

SEQ ID NO: 7 - protein, 349 aa, Synthetic construct
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEIIMMTGYEENAMWKLRADLPRIIPDAAIYYGYYYGMSALKIFAGFLFVDTWQYFLHRLMHMNKTLYKWFHSVHHELYVPYAYGALFNNPVEGFLLDTLGTGIAKTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

(19) United States
(12) Patent Application Publication
(10) Pub. No.: US 2026/0357310 A1
(43) Pub. Date: Nov. 12, 2026

(54) MUTANT OF YEAST SPHINGANINE C4-HYDROXYLASE SUR2 AND USE THEREOF

(71) Applicant: HANGZHOU VIABLIFE BIOTECH CO., LTD., Hangzhou, Zhejiang (CN)

(72) Inventors: Dayu YU, Hangzhou, Zhejiang (CN); Lei SUN, Hangzhou, Zhejiang (CN); Wei ZHANG, Hangzhou, Zhejiang (CN)

(21) Appl. No.: 19/118,323

(22) PCT Filed: Jul. 24, 2024
(86) PCT No.: PCT/CN2024/107341

§ 371 Date: Apr. 3, 2025

(30) Foreign Application Priority Data
Nov. 28, 2023 (CN) 202311597439.2

(51) Int. Cl. C12P 13/02; C12N 1/18; C12N 9/02; C12N 9/12; C12N 15/10; C12N 15/81; C12R 1/865

(52) U.S. Cl. CPC C12P 13/02; C12N 1/18; C12N 9/0071; C12N 9/1241; C12N 15/1024; C12N 15/1031; C12N 15/1058; C12N 15/81; C12Y 114/18; C12N 2800/102; C12R 2001/865

(57) ABSTRACT

The present disclosure discloses a mutant of yeast sphinganine C4-hydroxylase SUR2 and use thereof. The mutant is obtained by modifying enzyme gene through mutagenesis techniques such as error-prone PCR, followed by screening based on a specific modification purpose. The mutant can be used for efficient production of ceramide, and the production is characterized by simple fermentation conditions, ease of industrial implementation and control, and reduced generation of side products, thus facilitating subsequent ceramide extraction, reducing manufacturing costs, and possessing promising development prospects.

### CROSS-REFERENCE TO RELATED APPLICATION

The present disclosure claims priority to the Chinese patent application for invention with filing No. 202311597439.2 filed on Nov. 28, 2023 and entitled “MUTANT OF YEAST SPHINGANINE C4-HYDROXYLASE SUR2 AND USE THEREOF”, the contents of which are incorporated herein by reference in entirety.

### TECHNICAL FIELD

The present disclosure relates to the technical field of genetic engineering, and specifically, to a mutant of yeast sphinganine C4-hydroxylase SUR2 and use thereof.

### BACKGROUND ART

As a class of sphingolipid metabolites, ceramides are important components that constitute membranes of cells and organelles. Ceramide is present in all eukaryotic cells, and has an important regulation effect on cell differentiation, proliferation, apoptosis, aging and other vital activities. Ceramide is an intrinsic component of the human body, and plays a critical role in epidermal barrier function, not only preventing invasion of harmful substances, but also avoiding loss of moisture from the skin.

With the increase of ingredients in skincare products, ceramide used for moisturizing and anti-aging is getting increasingly popular, and the global ceramide market is growing steadily. Currently, ceramide products used in cosmetics are mainly extracted from plants. However, this method is limited by growth cycle of plants and environment, thus resulting in low yield, and obviously high cost at industrial level. Chemical synthesis involves complex steps, produces many by-products, has low synthesis efficiency, and is highly detrimental to the environment, making it difficult to achieve industrial-scale production. Researches find that Saccharomyces cerevisiae can directly produce ceramide, but the yield is relative low.

In view of this, the present disclosure is specifically proposed.

### SUMMARY

The present disclosure aims to providing a mutant of yeast sphinganine C4-hydroxylase SUR2 and use thereof, where the mutant is obtained by modifying enzyme gene through mutagenesis techniques such as error-prone PCR, followed by screening based on a specific modification purpose, and the mutant can be used for efficient production of ceramide.

The present disclosure is implemented as follows.

In the first aspect, the present disclosure provides a mutant of yeast sphinganine C4-hydroxylase SUR2, which is obtained by mutating wild-type yeast sphinganine C4-hydroxylase with amino acid sequence set forth in SEQ ID NO. 1, where a mutating mode is any one or a combination of more than one of the following (1)-(7):
(1) mutating M at position 153 in the amino acid sequence of the wild-type yeast sphinganine C4-hydroxylase to I, L or Y;
(2) mutating F at position 127 in the amino acid sequence of the wild-type yeast sphinganine C4-hydroxylase to Y;
(3) mutating M at position 124 in the amino acid sequence of the wild-type yeast sphinganine C4-hydroxylase to L;
(4) mutating Y at position 152 in the amino acid sequence of the wild-type yeast sphinganine C4-hydroxylase to F;
(5) mutating P at position 121 in the amino acid sequence of the wild-type yeast sphinganine C4-hydroxylase to I;
(6) mutating Y at position 123 in the amino acid sequence of the wild-type yeast sphinganine C4-hydroxylase to M; and
(7) mutating M at position 224 in the amino acid sequence of the wild-type yeast sphinganine C4-hydroxylase to K.

The yeast sphinganine C4-hydroxylase (yeast sphinganine C4-hydroxylase gene, SUR2) is present in yeast. In the present disclosure, by mutating the wild-type SUR2, the above SUR2 mutant is obtained, and can be used for synthesis of ceramide.

The amino acid sequence set forth in SEQ ID NO. 1 is SUR2 gene derived from Saccharomyces cerevisiae. All of SUR2 mutants obtained from wild-type SUR2 from other sources through any one or a combination of more than one of the above mutating modes (1)-(7) can be used for synthesis of ceramide.

In the present disclosure, the amino acid sequence refers to type and arrangement manner of amino acid residues that constitute a protein or polypeptide. An amino acid residue is generally represented by three letters or a single letter commonly used in the art. Those skilled in the art would be able to convert a three-letter amino acid sequence into a single-letter amino acid sequence, and vice versa. It should be noted that, regardless of the way of representation in the present disclosure, those skilled in the art could understand and convert between them. For example, alanine is represented by single letter A or three letters Ala; arginine by single letter R or three letters Arg; aspartic acid by single letter D or three letters Asp; cysteine by single letter C or three letters Cys; glutamine by single letter Q or three letters Gln; glutamate by single letter E or three letters Glu; histidine by single letter H and three letters His; isoleucine by single letter I and three letters Ile; glycine by single letter G and three letters Gly; asparagine by single letter N and three letters Asn; leucine by single letter L and three letters Leu; lysine by single letter K and three letters Lys; methionine by single letter M and three letters Met; phenylalanine by single letter F and three letters Phe; proline by single letter P and three letters Pro; Serine by single letter S and three letters Ser; threonine by single letter T and three letters Thr; tryptophan by single letter W and three letters Trp; tyrosine by single letter Y and three letters Tyr; valine by single letter V and three letters Val.

In some embodiments, the mutating mode of the SUR2 mutant is as follows:
mutating M at position 153 in the amino acid sequence set forth in SEQ ID NO. 1 to I, an amino acid sequence of such mutant being set forth in SEQ ID NO. 2; or
mutating M at position 153 in the amino acid sequence set forth in SEQ ID NO. 1 to I, and mutating M at position 124 to L, an amino acid sequence of such mutant being set forth in SEQ ID NO. 3; or
mutating M at position 153 in the amino acid sequence set forth in SEQ ID NO. 1 to L, and mutating Y at position 152 to F, an amino acid sequence of such mutant being set forth in SEQ ID NO. 4; or
mutating M at position 153 in the amino acid sequence set forth in SEQ ID NO. 1 to Y, and mutating Y at position 123 to M and mutating M at position 224 to K, an amino acid sequence of such mutant being set forth in SEQ ID NO. 5; or
mutating F at position 127 in the amino acid sequence set forth in SEQ ID NO. 1 to Y, an amino acid sequence of such mutant being set forth in SEQ ID NO. 6; or
mutating F at position 127 in the amino acid sequence set forth in SEQ ID NO. 1 to Y, and mutating P at position 121 to I, an amino acid sequence of such mutant being set forth in SEQ ID NO. 7.

That is, amino acid mutation of the mutant in the present disclosure is M153I or M153I/M124L or M153L/Y152F or F127Y or F127Y/P121I or M153Y/Y123M/M224K.

In the second aspect, the present disclosure provides a method for screening the above mutant, including: obtaining a target mutant through directed evolution by introducing nucleotide mutagenesis into SUR2 gene in vitro using an error-prone PCR technique, followed by multiple rounds of recombination and random mutagenesis.

Specifically, the screening method includes:
(1) purifying error-prone PCR amplification products using a DNA purification kit, followed by ligation with pESC-URA plasmid digested by restriction enzymes BamHI and HindIII, transferring ligation resultant into Saccharomyces cerevisiae competent cells using a lithium acetate method; plating transformed cells on a uracil dropout solid medium SD-URA plate, followed by inverted incubation at 25-30 °C for 2-5 days;
(2) by the same method as in step (1), performing multiple rounds of error-prone PCR with the mutant gene as a template to construct a mutant library;
(3) screening SUR2 mutant library:
inoculating transformants grown on the SD-URA plate into a 96-well deep-well plate containing SD-URA liquid medium, followed by incubation at 25-30 °C and 180-200 rpm for 18-24 hours; transferring cell suspension into a 96-well deep-well plate containing an induction medium, and inoculating wild-type Saccharomyces cerevisiae in parallel as control, followed by incubation at 25-30 °C and 600-800 rpm for 2-3 days; collecting the fermentation broth after 2-3 days of induced expression, extracting ceramide, analyzing yield thereof, and selecting strains with a significantly higher ceramide yield than the wild-type Saccharomyces cerevisiae under identical conditions as target strains; and
(4) shake-flask secondary screening for superior mutant strains:
dipping Saccharomyces cerevisiae strains with high ceramide yield from a mother board to streak colonies onto SD-URA plate, inverting the SD-URA plate in a 25-30 °C incubator to grow single colonies for 2-5 days; transferring activated single colony into SD-URA liquid medium, followed by incubation at 25-30 °C and 220-250 rpm for 16-20 hours overnight; inoculating cell suspension in SD-URA liquid into fermentation medium until initial OD₆₀₀=0.1-0.2, followed by incubation at 25-30 °C and 220-250 rpm for 2-3 days; after the incubation being ended, diluting 20-50 μL of the cell suspension with deionized water 40-100 times, and measuring absorbance at a wavelength of 600 nm in a spectrophotometer; moreover, taking 200-500 μL of fermentation broth, extracting ceramide, and determining ceramide yield.

Six strains with superior ceramide production efficiency are obtained by the above screening method, pESC-SUR2 expression plasmid therein is extracted using a yeast plasmid extraction kit, SUR2 nucleotide sequence is determined, and an amino acid sequence of SUR2 is extrapolated using a triplet codon. Modification modes of amino acid and relative activity for producing ceramide are listed in Table 1. Taking the relative activity of the control wild-type Saccharomyces cerevisiae for producing ceramide as 100%, the relative activity of engineered strains in the SUR2 mutant library for producing ceramide is determined based on analysis result of liquid phase detection.

The directed evolution employed in the present disclosure belongs to an irrational design, which refers to modification of enzyme gene through mutagenesis techniques such as error-prone PCR, followed by screening valuable natural enzymes based on a specific modification purpose. Currently, the directed evolution technology has been successfully applied to enhance enzymatic catalytic activity, improve substrate specificity, elevate thermal stability, and improve enantioselectivity and other aspects. The improved enzymatic activity in the present disclosure can be demonstrated by efficiency of producing ceramide by engineered strains expressing the mutant SUR2, and improved efficiency of producing ceramide by the engineered strains expressing the mutant SUR2 reflects improved activity of the mutant SUR2 in producing ceramide.

In the third aspect, the present disclosure provides a nucleic acid molecule encoding the above mutant.

In the fourth aspect, the present disclosure provides an expression vector containing the above nucleic acid molecule.

In the fifth aspect, the present disclosure provides a recombinant strain, which is constructed by introducing the above nucleic acid molecule into a host strain via plasmid or chromosomal integration through genetic engineering means.

In the present disclosure, the host strain includes Saccharomyces cerevisiae.

In some embodiments, the host strain is Saccharomyces cerevisiae PK2-1D.

In the sixth aspect, the present disclosure provides a cell catalyst including the above recombinant strain.

In the seventh aspect, the present disclosure provides use of the above mutant, biomaterial, recombinant strain or cell catalyst in ceramide production.

In the eighth aspect, the present disclosure provides a method for efficient synthesis of ceramide, wherein ceramide is synthesized by taking glucose as a substrate through whole-cell transformation of the above recombinant strain.

In some embodiments, a production system for the whole-cell transformation includes: 5-15 g/L glucose, 15-25 g/L galactose, 1-10 g/L yeast powder, 5-15 g/L peptone, 10-20 g/L α-cyclodextrin, 0.5-1.0 g/L CaCl₂), 0.5-1.0 g/L NH₄SO₄, 0.5-1.0 g/L KH₂PO₄, 0.1-0.5 g/L MgSO₄, 0.01-0.05 g/L FeSO4·7H2O, 10-50 mg/L niacin, and 1-20 g/L recombinant strain; and the whole-cell transformation is performed at a temperature of 25-30 °C for 2-3 days.

In the present disclosure, when synthesizing ceramide through the whole-cell transformation using the recombinant strain containing the above mutants, the method takes glucose as a substrate, galactose mainly as an inducer, and niacin as a precursor for synthesizing coenzyme NADPH.

Beneficial effects of the present disclosure are as follows.

In the present disclosure, the high-efficiency ceramide-producing SUR2 mutant is constructed through directed evolution, where starting from the ScSUR2 gene derived from Saccharomyces cerevisiae, the SUR2 mutant is obtained via the error-prone PCR method through directed evolution involving multiple rounds of recombination and random mutagenesis. Synthesis of ceramide with the mutant achieves significant increase in production yield, and the production is characterized by simple fermentation conditions, ease of industrial implementation and control, and reduced generation of side products, thus facilitating subsequent ceramide extraction, reducing manufacturing costs, and possessing promising development prospects.

### DETAILED DESCRIPTION OF EMBODIMENTS

In order to make objectives, technical solutions and advantages of embodiments of the present disclosure clearer, the technical solutions in the embodiments of the present disclosure will be described clearly and completely below. Embodiments, for which no concrete conditions are specified, are carried out according to conventional conditions or conditions recommended by manufactures. Where manufacturers of reagents or instruments used are not specified, they are conventional products commercially available.

High-efficiency ceramide-producing SUR2 mutants in the present disclosure are constructed through directed evolution, where starting from ScSUR2 gene (GenBank accession number U07171) SEQ ID NO. 1 derived from Saccharomyces cerevisiae, the SUR2 mutants are obtained via an error-prone PCR method through directed evolution involving multiple rounds of recombination and random mutagenesis. Amino acid sequences of the mutants contain amino acid mutations M153I, M153I/M124L, M153L/Y152F, F127Y, F127Y/P121I, and M153Y/Y123M/M224K. In terms of yield of ceramide produced by engineered strain expressing corresponding SUR2 mutants, these mutants improve the efficiency of producing ceramide, compared with control engineered strain expressing wild-type ScSUR2.

Host strain employed in the examples of the present disclosure is Saccharomyces cerevisiae PK2-1D.

Formulas of culture media involved in the present disclosure are as follows:
SD-URA liquid medium (6.7 g/L yeast nitrogen base (YNB); 2 g/L glucose, 0.76 g/L DO supplement-URA, pH 6.0);
SD-URA solid medium (6.7 g/L yeast nitrogen base (YNB); 2 g/L glucose, 0.76 g/L DO supplement-URA, 2 g/L agar powder, pH 6.0); and
fermentation medium (10 g/L glucose, 20 g/L galactose, 5 g/L yeast powder, 10 g/L peptone, 15 g/L α-cyclodextrin, 1 g/L CaCl₂), 1 g/L NH₄SO₄, 1 g/L KH₂PO₄, 0.43 g/L MgSO₄, 0.05 g/L FeSO4·7H2O, 50 mg/L niacin).

Induction medium and fermentation medium in the following examples have the same formulas.

High performance liquid chromatography (HPLC-ELSD) conditions are as follows: extracted ceramide is subjected to qualitative and quantitative analysis using Shimadzu LC-2050 liquid phase system, products are separated by reversed-phase chromatography SHIMADZU C8 (3 μm; 4.6×250 mm), with an eluent including 100% acetonitrile (solution A) and 0.06% (v/v) trifluoroacetic acid aqueous solution (solution B). Samples are diluted with 50% anhydrous ethanol, and a gradient elution program is as follows: elution with 60% solution A for 2 min, linear elution with 60%-75% solution A for 11 min, linear elution with 75%-93% solution A for 4 min, elution with 93% solution A for 3 min, and elution with 60% solution A for 5 min, where a flow rate is 1 mL/min, a column temperature is 40 °C, an injection volume is 10 μL, a drift tube temperature is 50 °C, drying gas (N₂) pressure is 350 kPa, and a filtration constant is 10.

### Example 1

The present example involved construction of a mutant library: high-efficiency ceramide-producing SUR2 mutants were constructed through directed evolution in the present example, where starting from ScSUR2 gene derived from Saccharomyces cerevisiae, the SUR2 mutants were obtained via an error-prone PCR method through directed evolution involving multiple rounds of recombination and random mutagenesis.

A nucleotide mutation method for the ScSUR2 gene in the present example included: introducing nucleotide mutagenesis into the ScSUR2 gene in vitro using an error-prone PCR technique:

Reaction conditions of the error-prone PCR were as follows

| | |
|---|---|
| 2.5 mM dNTP | 8 μL |
| Forward Primer (10 μM) | 2 μL |
| Reverse Primer (10 μM) | 2 μL |
| pYES2-ScSUR2 | 1 μL |
| Taq DNA polymerase | 1 μL |
| 10x Buffer | 10 μL |
| 10 mM Mn²⁺ | 3 μL |
| ddH₂O | 72 μL |
| total | 100 μL |

Upstream primer F:
5′-aggagaaaaaaccccggatccATGAACGTAACATCGAATGCAACT-3′;

Downstream primer R:
5′-gctagccgcggtaccaagcttTTATTTCTCTTTCTTTACTTCATTTTCA-3′

PCR amplification conditions: 94 °C 3 min; 94 °C. 1 min, 59 °C 1 min, 72 °C. 2 min, 30 cycles; 72 °C. 10 min;

Error-prone PCR amplification products were purified using a DNA purification kit, and then ligated with pESC-URA plasmid digested by restriction enzymes BamHI and HindIII. Ligation resultant was transformed into Saccharomyces cerevisiae competent cells using a lithium acetate method. Transformed cells were plated on a uracil dropout solid medium SD-URA plate, and incubated at 30 °C for 2 days.

By the same method as above, multiple rounds of error-prone PCR were performed with the mutant gene as a template to construct the mutant library.

### Example 2

The present example involved mutant library screening: transformants grown on the SD-URA plate were inoculated with a sterilized toothpick into a 96-well deep-well plate containing 1 mL of SD-URA liquid medium, incubated on a shaker at 30 °C and 800 rpm for 24 hours. Cell suspension was transferred into a 96-well deep-well plate containing an induction medium, and Saccharomyces cerevisiae PK2-1D/pESC-ScSUR2-WT was inoculated in parallel as control, and incubated on a shaker at 30 °C and 800 rpm for 2 days. After fermentation was ended, 500 μL of fermentation broth from the 96-well plate that had induced to express for 2 days was taken, followed by ultrasonic disruption, then ceramide was extracted with petroleum ether, yield of ceramide was analyzed using a high performance liquid chromatography HPLC-evaporative light scattering detector (ELSD). Under identical conditions, the control engineered strain PK2-1D/pESC-ScSUR2-WT only produced trace amounts of ceramide, and strains with a significantly higher ceramide yield than the control engineered strain PK2-1D/pESC-ScSUR2-WT under identical conditions were chosen as target strains.

### Example 3

The present example involved shake-flask secondary screening for superior mutant strains. Saccharomyces cerevisiae strains with high ceramide yield were dipped from a mother board by a toothpick to streak colonies onto SD-URA plate, and the SD-URA plate was inverted in a 30 °C incubator to grow single colonies for 2 days. Activated single colony was transferred into 3 mL of SD-URA liquid medium, and incubated at 30 °C and 250 rpm for 16 hours overnight. Cell suspension in SD-URA liquid was inoculated into 25 mL of induction medium until initial OD₆₀₀=0.2, and incubated at 30 °C and 250 rpm for 3 days. After the incubation was ended, 50 μL of the cell suspension was diluted 40 times with deionized water, and measured for absorbance at a wavelength of 600 nm in a spectrophotometer. Moreover, 500 μL of fermentation broth was collected and subjected to ultrasonic disruption, then ceramide was extracted with petroleum ether, and after a sample was filtered through a 0.22 μm filter membrane, ceramide yield was measured by HPLC-ELSD.

Six strains with superior ceramide production efficiency were screened out from the mutant library constructed via the error-prone PCR method, pESC-SUR2 expression plasmid therein was extracted using a yeast plasmid extraction kit, SUR2 nucleotide sequence was determined, and an amino acid sequence of SUR2 was extrapolated using a triplet codon. Modification modes of amino acid and relative activity for producing ceramide are listed in Table 1. Taking the relative activity of the control engineered strain PK2-1D/pESC-ScSUR2-WT for producing ceramide as 100%, the relative activity of the engineered strains in the SUR2 mutant library for producing ceramide was determined based on analysis result of liquid phase detection.

Table 1 Sequencing Results of High-Yield Ceramide-Producing Mutants Nos. SUR2 1-6 and Yield of Plant Ceramide

| | Amino acid change | Plant ceramide | Increase fold of plant | Amino acid |
|---|---|---|---|---|
| Strain No. | mode | yield | ceramide yield | sequence No. |
| ScSUR2-WT | | 13.02 mg/L | | SEQ ID NO. 1 |
| ScSUR2-1 | M153I | 21.37 mg/L | 164% | SEQ ID NO. 2 |
| ScSUR2-2 | M153I/M124L | 25.64 mg/L | 196% | SEQ ID NO. 3 |
| ScSUR2-3 | M153L/Y152F | 40.11 mg/L | 308% | SEQ ID NO. 4 |
| ScSUR2-4 | F127Y | 32.06 mg/L | 246% | SEQ ID NO. 5 |
| ScSUR2-5 | F127Y/P121I | 36.22 mg/L | 278% | SEQ ID NO. 6 |
| ScSUR2-6 | M153Y/Y123M/M224K | 44.91 mg/L | 345% | SEQ ID NO. 7 |

Note: the yield of plant ceramide in Table 1 was total yield of ceramide 3 and ceramide 3B, where a mass ratio of ceramide 3 to ceramide 3B was 3:1.

As evidenced from Table 1, the mutants obtained in the present disclosure included amino acid mutations M153I, M153I/M124L, M153L/Y152F, F127Y, F127Y/P121I, and M153Y/Y123M/M224K. The yield of the mutants for producing the plant ceramide was 1.64-3.45 times of that of the wild type.

The above-mentioned are merely for preferred embodiments of the present disclosure, but are not intended to limit the present disclosure. For those skilled in the art, various modifications and changes could be made to the present disclosure. Any modifications, equivalent substitutions, improvements and so on made within the spirit and principle of the present disclosure should be covered within the scope of protection of the present disclosure.

### INDUSTRIAL APPLICABILITY

The mutant of yeast sphinganine C4-hydroxylase SUR2 provided in the present disclosure is obtained by modifying enzyme gene through mutagenesis techniques such as error-prone PCR, followed by screening based on a specific modification purpose, and the mutant can produce ceramide efficiently.

1. A mutant of yeast sphinganine C4-hydroxylase SUR2, which is obtained by mutating wild-type yeast sphinganine C4-hydroxylase with amino acid sequence set forth in SEQ ID NO. 1, wherein a mutating mode is any one or a combination of more than one of the following (1)-(7):
(1) mutating M at position 153 in the amino acid sequence of the wild-type yeast sphinganine C4-hydroxylase to I, L or Y;
(2) mutating F at position 127 in the amino acid sequence of the wild-type yeast sphinganine C4-hydroxylase to Y;
(3) mutating M at position 124 in the amino acid sequence of the wild-type yeast sphinganine C4-hydroxylase to L;
(4) mutating Y at position 152 in the amino acid sequence of the wild-type yeast sphinganine C4-hydroxylase to F;
(5) mutating P at position 121 in the amino acid sequence of the wild-type yeast sphinganine C4-hydroxylase to I;
(6) mutating Y at position 123 in the amino acid sequence of the wild-type yeast sphinganine C4-hydroxylase to M; and
(7) mutating M at position 224 in the amino acid sequence of the wild-type yeast sphinganine C4-hydroxylase to K.

2. The mutant according to claim 1, wherein the mutating mode of the mutant is:
mutating M at position 153 in the amino acid sequence set forth in SEQ ID NO. 1 to I; or
mutating M at position 153 in the amino acid sequence set forth in SEQ ID NO. 1 to I, and mutating M at position 124 to L; or
mutating M at position 153 in the amino acid sequence set forth in SEQ ID NO. 1 to L, and mutating Y at position 152 to F; or
mutating M at position 153 in the amino acid sequence set forth in SEQ ID NO. 1 to Y, and mutating Y at position 123 to M and mutating M at position 224 to K; or
mutating F at position 127 in the amino acid sequence set forth in SEQ ID NO. 1 to Y; or
mutating F at position 127 in the amino acid sequence set forth in SEQ ID NO. 1 to Y, and mutating P at position 121 to I.

3. A method for screening the mutant according to claim 1, wherein a target mutant is obtained through directed evolution by introducing nucleotide mutagenesis into wild-type yeast sphinganine C4-hydroxylase gene in vitro using an error-prone PCR technique, followed by recombination and random mutagenesis.

4. A nucleic acid molecule encoding the mutant according to claim 1.

5. An expression vector comprising the nucleic acid molecule according to claim 4.

6. A recombinant strain, wherein the recombinant strain is constructed by introducing the nucleic acid molecule according to claim 4 into a host strain via plasmid or chromosomal integration through genetic engineering means, wherein the host strain comprises Saccharomyces cerevisiae.

7. A cell catalyst comprising the recombinant strain according to claim 6.

8. (canceled)

9. A method for efficient synthesis of ceramide, wherein the ceramide is synthesized by taking glucose as a substrate through whole-cell transformation of the recombinant strain according to claim 6.

10. The method for efficient synthesis of ceramide according to claim 9, wherein a production system for the whole-cell transformation comprises:
5-15 g/L glucose, 15-25 g/L galactose, 1-10 g/L yeast powder, 5-15 g/L peptone, 10-20 g/L α-cyclodextrin, 0.5-1.0 g/L CaCl₂, 0.5-1.0 g/L NH₄SO₄, 0.5-1.0 g/L KH₂PO₄, 0.1-0.5 g/L MgSO₄, 0.01-0.05 g/L FeSO4·7H2O, 10-50 mg/L niacin, and 1-20 g/L recombinant strain; and
the whole-cell transformation is performed at a temperature of 25-30 °C for a duration of 2-3 days.

11. A method for screening the mutant according to claim 2, wherein a target mutant is obtained through directed evolution by introducing nucleotide mutagenesis into wild-type yeast sphinganine C4-hydroxylase gene in vitro using an error-prone PCR technique, followed by recombination and random mutagenesis.

12. The nucleic acid molecule according to claim 4, wherein the mutating mode of the mutant is:
mutating M at position 153 in the amino acid sequence set forth in SEQ ID NO. 1 to I; or
mutating M at position 153 in the amino acid sequence set forth in SEQ ID NO. 1 to I, and mutating M at position 124 to L; or
mutating M at position 153 in the amino acid sequence set forth in SEQ ID NO. 1 to L, and mutating Y at position 152 to F; or
mutating M at position 153 in the amino acid sequence set forth in SEQ ID NO. 1 to Y, and mutating Y at position 123 to M and mutating M at position 224 to K; or
mutating F at position 127 in the amino acid sequence set forth in SEQ ID NO. 1 to Y; or
mutating F at position 127 in the amino acid sequence set forth in SEQ ID NO. 1 to Y, and mutating P at position 121 to I.
